(19) United States
(12) Patent Application Publication
(10) Pub. No.: US 2019/0392946 A1
(43) Pub. Date: Dec. 26, 2019

(54) RESIDENT COMMUNITY MANAGEMENT SYSTEM

(71) Applicant: Atria Senior Living, Inc., Louisville, KY (US)

(72) Inventors: Bryan Chamberlain, Louisville, KY (US); Joshua Wiscaver, Louisville, KY (US); Amanda Marburger, Louisville, KY (US); Robert Azinger, Louisville, KY (US); Michael Gentry, Louisville, KY (US); Kathy Lewis, Louisville, KY (US); Carol Griffin, Louisville, KY (US)

(21) Appl. No.: 16/179,396

(22) Filed: Nov. 2, 2018

Related U.S. Application Data
(62) Division of application No. 16/013,579, filed on Jun. 20, 2018.

(51) Int. Cl. G16H 40/20; G16H 10/20; G06Q 50/16; G16H 20/10; G16H 10/60

(52) U.S. Cl. CPC G16H 40/20; G16H 10/20; G16H 10/60; G16H 20/10; G06Q 50/163

(57) ABSTRACT

Systems and methods are provided for managing a plurality of resident communities having a plurality of rooms for occupancy, the system accessible by a plurality of individual users. The system may include a memory device that stores a set of instructions for operating the system. The system further stores room data relevant to the resident communities, assigns room data to individual rooms based on historical occupancy rates and rental price rates, and compares individual room data by one of a plurality of room groupings to calculate a recommended price for each room based on a weighted room data average.

### FIELD OF THE INVENTION

The present system is related generally to a system for management of a residential facility living environment. More specifically, the system described herein relates to a smart system for tracking, monitoring, and interacting with a plurality of residents located in a plurality of resident communities. The invention also relates to systems and apparatus for providing customized resident services, health assessments, incident reports and billing systems customized to meet the needs of individual residents and residential facility operators.

### BACKGROUND OF THE INVENTION

Recently, residential living communities have proliferated in many geographical areas as the large number of “baby boomers” have attained retirement age. As often happens with aging populations, physical limitations and acute and chronic medical issues often cause people to seek out residential living communities, often called “retirement communities”, where they have access to assistance in some activities of daily living and where the day-to-day tasks associated with living in their own home are no longer required.

Residential communities come in many different varieties offering differing levels of care and assistance to their residents. Furthermore, many residential communities offer a broad selection of care and assistance options for residents that are attempts at meeting each residents' needs. Of course residents in many communities are widely different. Some require frequent care and assistance with activities of daily living, while others reside in the community wholly unassisted. Thus, there are many operational challenges to operating a residential community that houses residents with a broad variety of needs.

Residents having larger or more luxurious living spaces typically pay higher monthly rates than those occupying more modest accommodations. Additionally, room or facility lease rates are highly market dependent, varying widely based on the room size and location within the facility, the location of the facility, comparable facility rates in the surrounding geographical area, and generally the cost of living in the area, or in areas having similar market attributes. As a further consideration, residents requiring considerable assistance or oversight with daily living activities will pay additional fees based on levels of care and/or medication management depending upon the amount of assistance required. Furthermore, in the case of many residents, some nursing or medical care may be required on a periodic or continuous basis, which adds further cost to the residential community provider. Obviously, accounting for and tracking all the various different service levels, care levels, rental rates and room information data required to efficiently operate a typical residential community is a monumental task.

Currently, a variety of off-the-shelf business enterprise solutions are purchased and used by some residential care facility operators. Many of these off the shelf solutions for residential facility management lack various features that enable one to efficiently operate a residential care facility. Property management and lease management systems such as Real Pages™ and resident and lead management systems such as Move-n™ are available to manage some aspects of residential living arrangements, but these prior art systems lack sophisticated tools for managing and tracking the variety of variables required for a residential care facility operator.

Yardi® further utilizes commercial systems for lease management and assessment and task management, but as with other prior art systems the ability to provide sophisticated assessment tools, lead tracking, self-guided assessments, occupancy analysis, resident care profiles, and billing tools are largely absent. For example, the ability to track each resident's care, from entry into a facility to discharge, and to organize and coordinate facility sales and marketing management, incident reporting, and resident life are largely missing from the prior art.

Accordingly, a need exists in the art for a system of tracking and managing tools for managing a residential community at a resident level, a facility level, and a regional level and further for coordinating resources and data within each facility to efficiently allocate business resources at a national or business-wide level.

### SUMMARY OF THE INVENTION

This disclosure is directed generally to systems, methods and apparatus that provide a residential care management system to provide information to community operators and care directors and employees in a plurality of residential care facilities. In accordance with some aspects of the invention, a residential community management system is provided that utilizes a dedicated computer network and a database that is employed to collect, analyze, and store data related to the operation and management of a plurality of residential care facilities and to the residents therein. The system employs a plurality of web servers to allow individual users to interact with and modify system attributes related to a broad array of data required to be tracked and collected in the operation of a care facility, including but not limited to regulatory compliance, care management, sales, community census and billing, resident safety and incident reporting, performance metrics, and marketing. These data categories are collected and displayed for each individual residential community and may be concatenated to provide a management system that provides management tools that may be customized for each resident, community and region within the system. The system may further include a plurality of user controlled external devices such as a smartphone, a smart watch, a tablet, or any device that utilizes wireless network communication, or any other communication system, wired or wireless.

In some embodiments, the systems and methods described herein may each include one or more of the following features. A system that tracks performance metrics of a plurality of residential care facilities that is accessible by a plurality of users at various levels of accessibility. The system described herein includes the ability to track a plurality of individual rooms and assign data attributes thereto based on historical occupancy rates, historical price rates, and market rates. In further embodiments, the system includes the ability to compare individual rooms by a plurality of room groupings and calculated recommended prices for individual rooms based on weighted averages of the room data stored in the database.

Furthermore, the system disclosed herein provides residential care facility room comparisons by a plurality of room groupings, including but not limited to regions (geographical groupings), clusters of communities, based on factors such as pricing level, proximity, service level, and physical attributes, room types, and markets. Additional exemplary embodiments may include the calculation of recommended room pricing based on a weighted room occupancy rate index, a market position index, and a historical rate index and may include individual rate maximums and minimums as a feature of the recommended rate.

In accordance with further embodiments and aspects of the residential facility management system, a plurality of room occupancy metrics may be established based upon the number of tours given or provided over a time period for a room or grouping thereof, the number of inquiries about a room or grouping in the time period, and the number of residents moving in (move-ins) in the time period. Thus, the invention may provide a user with an inquiry-to-tour ratio and a tour-to move-in ratio for selected room groupings based on geographical region, market area, business operation unit groupings, and room attribute groupings. Room attribute groupings typically include room features such as one or two bedroom, studio, patio, and various other features typical of residential facility rooms.

In various aspects of the invention, a community dashboard is provided for each residential community in the system. The community dashboard provides a plurality of community and resident metrics to a user, including a census detailing the number of residents occupying that specific community and the number of vacant rooms. A sales and marketing interface is also provided that includes the ability for a user to select and view recent sales leads and tours as well as follow up actions for the sales personnel. In some aspects and embodiments, the invention produces sales tools including but not limited to a lead manager, a referral manager for outside referrals, a bonus or incentive pay forecaster, a community census tracker, a reservation system, a sales activity monitor, and a communications interface for producing documents and electronic communications germane to a specific residential community.

In various other aspects and embodiments, the system provides an assessment tool for each resident in a residential community. The assessment tool may be used by resident care staff or sales staff to consider and collect data regarding the health and care requirements of each resident in a given community. The assessments produced by the various implementations are used to produce an acuity score, which represents a general level of care required for an individual resident. Based upon the periodic assessments provided to each resident, the acuity score, and the room leased by each resident, a care profile of each resident may be accurately formulated and automatically updated upon a change in status. Further, the assessment tool provided herein includes a prospective resident embodiment wherein a person considering residency may be initially assessed by a member of the sales staff, or alternatively provide a self-assessment to determine approximate costs for residential care. These assessments are then utilized to produce a service plan for each resident, based closely upon the care profile produced by the implementations disclosed herein, that provides detailed pricing information for each individual resident.

The various implementations of the system further provide the ability to provide wireless communications from a residential care staff member to the system, so that various system attributes and data measurements may be provided to the system in real time, as the staff performs their daily tasks. This feature of the invention assures that enhanced care that may be required by changed circumstances for a particular resident is instantly updated in the system, such that care profiles and service plans accurately reflect any changes required for a given resident.

The systems and methods discussed herein further include embodiments for tracking and reporting resident incidents by resident, community, and region. The incident reporting system disclosed herein includes producing required regulatory reporting forms to appropriate residential community personnel. The implementations further provide tools for tracking instances of resident accidents, falls, health-related incidents, theft incidents, elopement incidents, and medicinal or prescription issues, and providing management notification of each incident reported. In some implementations and aspects, the system of the invention may track resident preferences in a wide variety of categories are also recorded and provided to care staff, including but not limited to resident food preferences, spiritual preferences, entertainment preferences and other resident preferences.

As used herein for purposes of the present disclosure, the term “wireless communication” generally describes apparatus and systems relating to the wireless transmission of a signal. Any of a wide variety of wireless transmission devices and communications protocols may be employed in the system of the invention, including analog and digital transmission systems. Exemplary but non-limiting wireless transmitters that may form a part of the invention include radio transmitters, cellular transmitters, LTE and LTE advanced systems, ZigBee™, Wi-Fi, and Bluetooth transmitters. Additionally, a plurality of wireless network and transmission systems may be employed without departing from the scope of the invention, including, but not limited to, wireless personal area networks, local area networks, mesh networks, metropolitan area and global area networks.

The term “processor” or alternatively “controller” is used herein generally to describe various apparatus relating to the operation of one or more computers, web servers, or databases. A processor can be implemented in numerous ways (e.g., such as with dedicated hardware) to perform various functions discussed herein. A “processor” is one example of a controller which employs one or more microprocessors that may be programmed using software instructions (e.g., microcode) to perform various functions discussed herein. A controller may be implemented with or without employing a processor, and also may be implemented as a combination of dedicated hardware to perform some functions and a processor (e.g., one or more programmed microprocessors and associated circuitry) to perform other functions. Examples of controller components that may be employed in various embodiments of the present disclosure include, but are not limited to, conventional microprocessors, application specific integrated circuits (ASICs), and field-programmable gate arrays (FPGAs).

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present disclosure discussed herein. The terms “program” or “computer program” or “instructions” are used herein in a generic sense to refer to any type of computer code (e.g., software or microcode) that can be employed to program one or more processors or controllers.

The term “user interface” as used herein refers to an interface between a user or operator and one or more devices that enables interaction between the user and the device(s). Examples of user interfaces that may be employed in various implementations of the present disclosure include, but are not limited to, switches, potentiometers, buttons, dials, sliders, a mouse, keyboard, keypad, various types of game controllers (e.g., joysticks), track balls, display screens, various types of graphical user interfaces (GUIs), smartphones, watches, tablets, personal computing platforms, touch screens, microphones and other types of sensors that may receive some form of human-generated stimulus and generate a signal in response thereto. Furthermore, user interfaces can encompass interactive web pages and other user prompts, whether provided on stand alone computing platforms or mobile devices.

The term “communications link” is generally meant to include in digital or other communication with any other part of the system via a wireless or wired communication protocol. A communication link may be between two devices or components and may be accomplished by a separate networking system. Communication links may be provided to transfer data between a web server, a database, a computer, a mobile or handheld device, or any other control system, a consumer operated external device, a wireless local area network (WLAN), or any other communication system. The communication links disclosed and described in this specification may be integrated within various system components or alternatively may be separate electronic systems.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Before explaining exemplary embodiments consistent with the present disclosure in detail, it is to be understood that the disclosure is not limited in its application to the details of constructions and to the arrangements set forth in the following description or illustrated in the drawings. The disclosure is capable of embodiments in addition to those described and is capable of being practiced and carried out in various ways. Also, it is to be understood that the phraseology and terminology employed herein, as well as in the abstract, are for the purpose of description only and should not be regarded as limiting.

The accompanying drawings, which are incorporated and form a part of the specification illustrate exemplary, but non-limiting, embodiments of the disclosure, and together with the description, serve to explain the principles of the disclosure.

Those skilled in the art will appreciate that the inventive concepts and principles upon which the disclosure is based may readily be utilized as a basis for designing other structures, systems, methods, and articles of manufacture for implementing the purposes of the present disclosure. Accordingly, the claims appended hereto should be construed to include such equivalent constructions without departing from the spirit and scope of the invention herein disclosed.

### BRIEF DESCRIPTION OF THE DRAWING FIGURES

FIG. 1 illustrates an exemplary system structure for implementing embodiments and features of the present disclosure;

FIG. 2 illustrates an exemplary block diagram of a system for implementing embodiments and features of the present disclosure;

FIG. 3 illustrates an exemplary residential community dashboard for implementing embodiments and features of the present disclosure;

FIG. 4 illustrates an exemplary residential community dashboard for implementing embodiments and features of the present disclosure;

FIG. 5 illustrates an exemplary room census system for implementing embodiments and features of the present disclosure;

FIG. 6 illustrates an exemplary system for providing self-assessments by potential residents in accordance with embodiments and features of the present disclosure;

FIG. 7 illustrates an exemplary survey questions for conducting self-assessments by potential residents in accordance with embodiments and features of the present disclosure;

FIG. 8 illustrates an exemplary survey questions for conducting self-assessments by potential residents in accordance with embodiments and features of the present disclosure;

FIG. 9 illustrates an exemplary care profile interface for potential residents in accordance with embodiments and features of the present disclosure;

FIG. 10 illustrates an exemplary assessment model for community residents in accordance with embodiments and features of the present disclosure;

FIG. 11 illustrates an exemplary assessment model for community residents in accordance with embodiments and features of the present disclosure;

FIG. 12 illustrates an exemplary assessment model for community residents in accordance with embodiments and features of the present disclosure;

FIG. 13 illustrates an exemplary nested question in accordance with embodiments and features of the present disclosure;

FIG. 14 illustrates an exemplary nested question in accordance with embodiments and features of the present disclosure;

FIG. 15 illustrates an exemplary nested question in accordance with embodiments and features of the present disclosure;

FIG. 16 illustrates an exemplary set of interpretive guidelines in accordance with embodiments and features of the present disclosure.

FIG. 17A illustrates an exemplary care profile for community residents in accordance with embodiments and features of the present disclosure;

FIG. 17B illustrates an exemplary care profile for community residents in accordance with embodiments and features of the present disclosure;

FIG. 17C illustrates an exemplary care profile for community residents in accordance with embodiments and features of the present disclosure;

FIG. 18A illustrates an exemplary service plan for community residents in accordance with embodiments and features of the present disclosure;

FIG. 18B illustrates an exemplary service plan for community residents in accordance with embodiments and features of the present disclosure;

FIG. 18C illustrates an exemplary service plan for community residents in accordance with embodiments and features of the present disclosure;

FIG. 18D illustrates an exemplary service plan for community residents in accordance with embodiments and features of the present disclosure;

FIG. 18E illustrates an exemplary service plan for community residents in accordance with embodiments and features of the present disclosure;

FIG. 18F illustrates an exemplary service plan for community residents in accordance with embodiments and features of the present disclosure;

FIG. 19 illustrates an exemplary incident report for community residents in accordance with embodiments and features of the present disclosure;

FIG. 20 illustrates an exemplary incident report for community residents in accordance with embodiments and features of the present disclosure;

FIG. 20B illustrates an exemplary incident report for community residents in accordance with embodiments and features of the present disclosure;

FIG. 20C illustrates an exemplary incident report for community residents in accordance with embodiments and features of the present disclosure;

FIG. 20D illustrates an exemplary incident report for community residents in accordance with embodiments and features of the present disclosure;

FIG. 20E illustrates an exemplary incident report for community residents in accordance with embodiments and features of the present disclosure;

FIG. 20F illustrates an exemplary incident report for community residents in accordance with embodiments and features of the present disclosure; and

FIG. 21 illustrates an exemplary incident dashboard for community residents in accordance with embodiments and features of the present disclosure.

### DETAILED DESCRIPTION OF EXEMPLARY EMBODIMENTS

Reference will now be made in detail to the present embodiments of the disclosure, certain non-limiting examples of which are illustrated in the accompanying drawing Figures.

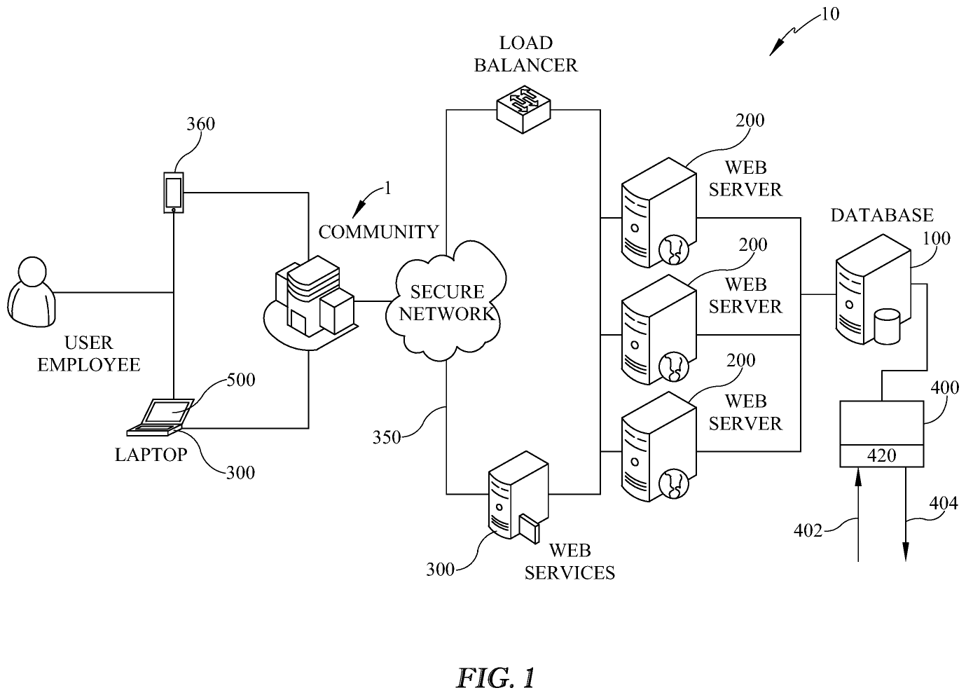

Referring to FIG. 1, and in accordance with some embodiments of the invention, a system 10 for managing and monitoring a plurality of residential communities 1 each having at least one, but preferably a plurality of residents occupying the communities 1 is shown. In various embodiments, system 10 may include at least one database 100, operatively coupled to and communicating with a plurality of web servers 200. Web servers 200 are coupled to and communicate with each other and a plurality of web-enabled computers 300, via a secure network 350. The secure network provides system 10 access to a plurality of computers 300 through each residential community via a conventional login screen, wherein a user may enter a login name and password to access the secure network via conventional internet communications protocols, as is known in the art.

Each database 100, server 200, and computer 300 may be equipped with a processor or controller 400, for example a microcontroller having a plurality of electrical signal inputs 402 and outputs 404 that may be operatively coupled to various components of system 10 described herein. Processors 400 may include concomitant data storage memory 420, both RAM and ROM, and further may also be operatively coupled to additional storage memory 420 for storing and using data acquired during operation of system 10. Memory 420 can include a number of separate memories including a main random access memory (RAM) for storage of instructions and data during processor 400 operation and a read only memory (ROM) in which fixed instructions may be stored.

Furthermore, in some embodiments and implementations a handheld or mobile computing device 360 may also be provided in data communication with secure network to provide a user the ability to access and interact with system 10 while moving around a residential community. Exemplary but non-limiting mobile devices include smartphones, tablet computers, smart watches, and other devices capable of internet access utilizing a web browser or similar tools. As will be seen herein below, mobile computing devices 360 provide an operator the ability to access a wide array of system 10 attributes required for efficient system 10 operation.

In various aspects of the invention system 10 includes a user interface 500 that may include a display screen or screens that are provided to a user through computers 300, or alternatively a touch sensitive screen, a plurality of buttons or option selector switches, a keypad, a keyboard, mouse, or a touch sensitive pad to enable a user to configure system 10 for use and to access system 10 attributes via computers 300. User interfaces 500 may further include a speaker or audible alarm to alert or notify a user of certain prompts or actions. One of ordinary skill in the art will recognize that a wide variety of user interfaces may be employed in conjunction with the exemplary embodiments disclosed herein without departing from the scope of the invention. In various aspects and embodiments of the invention, user interface 500 will typically be provided as a series of displays provided through a web browser interface, although various non-limiting user interfaces may be employed without departing from the scope of the invention.

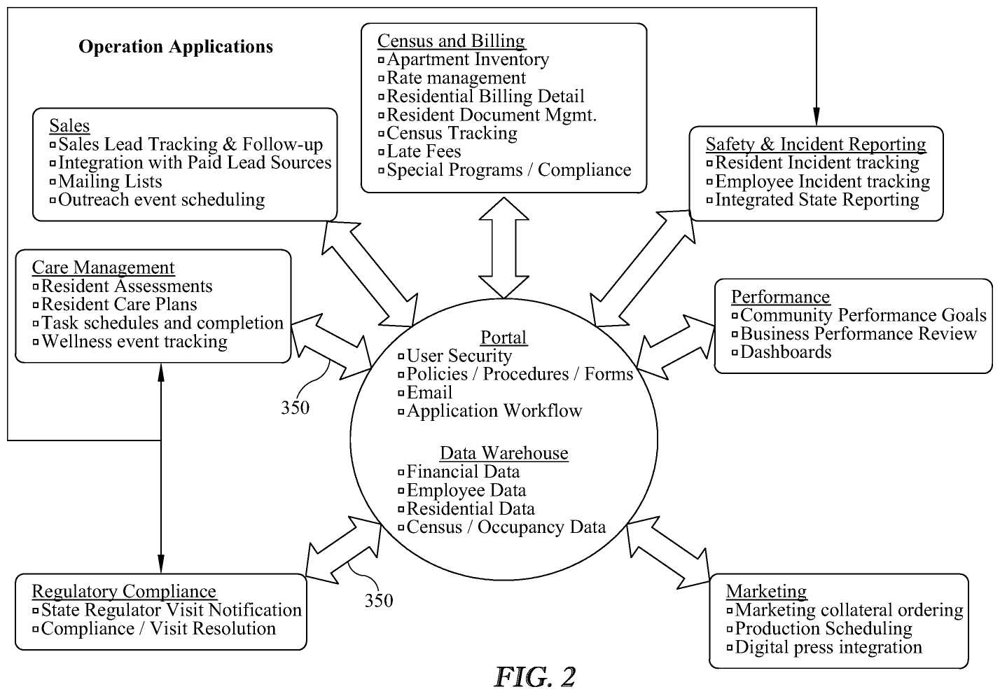

Referring now to FIGS. 1 and 2, and in accordance with certain embodiments, system 10 provides a user the ability to access, view, and modify a vast array of data required to track and manage a residential community that fall generally into the categories of regulatory compliance, care management, sales, community census and billing, resident safety and incident reporting, performance metrics, and marketing. These general data categories are provided for each individual residential community and may be collected and concatenated to provide a management system 10 that provides management tools that may be customized for each resident, community and region with system 10, as well as the entire national and/or company-wide network 350 of residential communities that form a part of system 10. All the data relevant to each resident and community is input by users through operation of system 10 via a browser interface, and stored in database 100 memory for further use. As will be discussed in further detail below, each of these data categories include a plurality of sub-categories that are useful in operation of system 10.

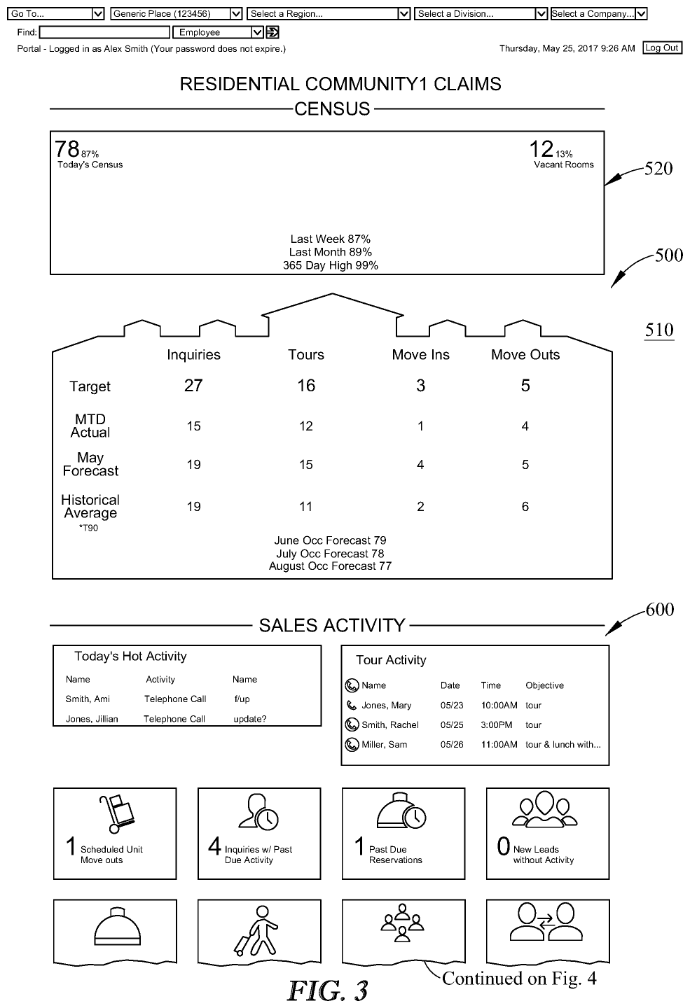

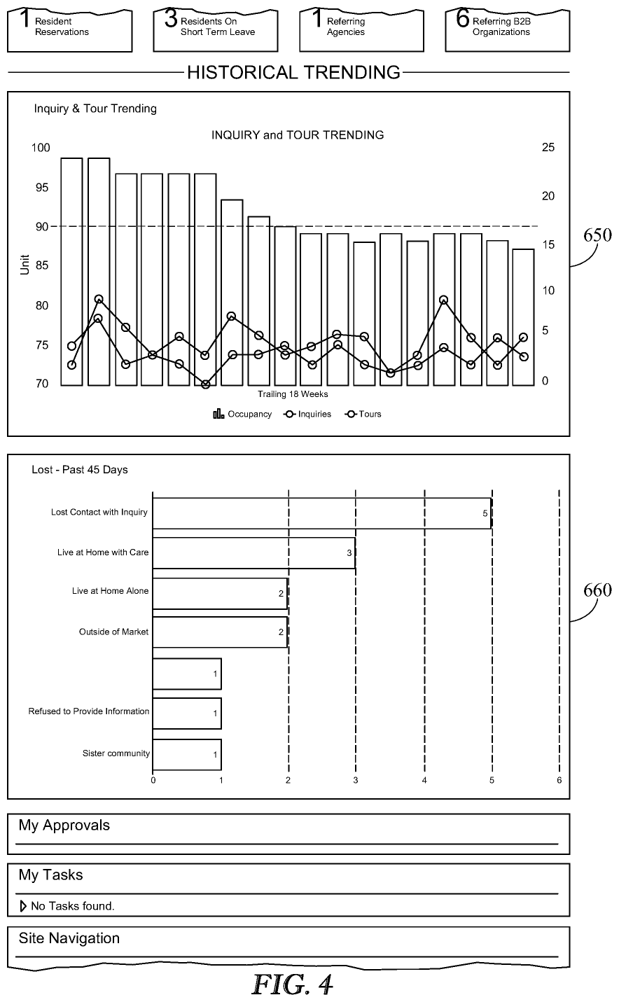

As best seen in FIGS. 3 and 4 in accordance with some aspects of the invention system 10 utilizes an instruction set, for example one produced by known programming methods and languages, that produces and displays a series of user displays having a plurality of embedded links therein that permits a user to navigate between various system 10 features, and to select and produce system 10 outputs representative of resident, community, or regional data attributes. As will be seen from the illustrations and embodiments herein, a wide array of user directed outputs can be obtained by operation of system 10 that enable efficient management of multiple residential communities encompassing a plurality of residents, while enabling a user to provide customized and individual care plans for individual residents.

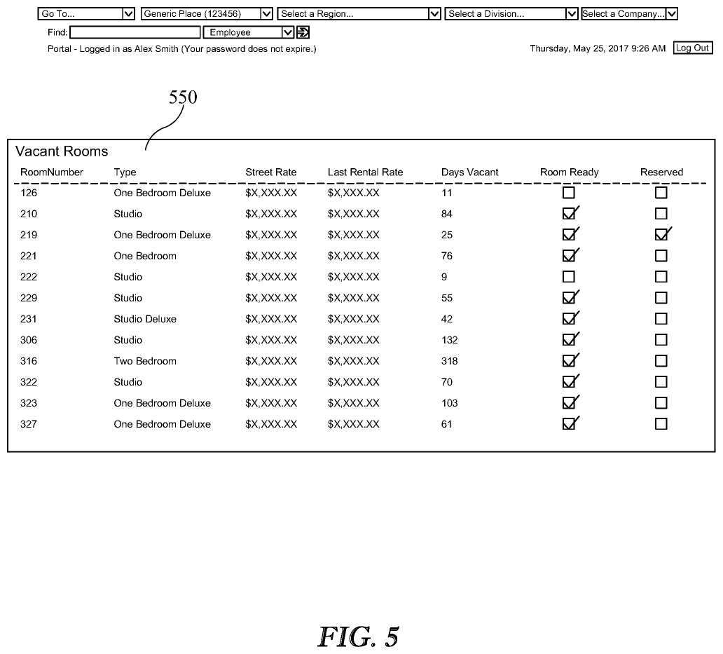

FIGS. 3 and 4 depict an exemplary but non-limiting user interface 500 for a residential care dashboard 510 for an individual residential community that includes a resident census 520 depicting the number of residents and the number of rooms leased in the facility, the number of vacant rooms, and the percentage of occupancy for the day, last week, last month and last year. Selecting the vacant rooms link navigates a user to a vacant room list 550 on a separate page, depicted here in FIG. 5, that provides an instant snapshot of available rooms by room number, and further shows the room type, street rate, last rental rate, number of days the room has been vacant, and whether the room is ready to be leased or is reserved. This feature of the invention permits a user, for example a facility operator or salesperson, to rapidly access all available rooms and rates for any residential community in system 10.

Below the resident census 520 there is displayed facility inquiry and move in/move out data for each residential community. The number of inquiries for prospective residents, number of tours, number of move ins, and number of move outs are displayed in each of four business target areas, Target amounts, MTD actual, Monthly forecast, and historical average. On residential care dashboard 510 a display of sales activity 600 is further provided, with lists of “hot” leads, tour schedules, move-outs schedules, recent inquires, past due reservations, new leads, current reservations, residents on short term leave, referring agencies, and referring business to business organizations. Selecting any of these sales icons displays an additional user interface that details the activity in the respective sales category so that an operator or user may review data provided in the individual category at a granular level.

In various embodiments and implementations, resident care dashboard 510 further includes a historical trending interface 650, depicting a graphical representation of inquiries and tours, and their respective trend lines, over a predetermined time period for a given residential care facility. This feature of the invention provides an operator with a rapid and accurate view of the utilization trend for a specific residential community 1, and permits managers of multiple facilities or regional groupings of facilities to view aggregate trends of community occupancy and thereby make informed decisions as to resource allocation and sales and marketing efforts.

In various implementations and embodiments, the invention provides a variety of assessment tools to assess and track the acuity level of each individual resident and potential resident of a facility. In the context of the present application and embodiments of the invention, the term “acuity level” may be construed to encompass all attributes of a person's physical, mental, and emotional condition at the time the assessment of the individual takes place. The system 10 of the invention provides several systems and methods for assessment of residents and potential residents that permits efficient care scheduling, staffing, and concomitant pricing based on the individual facility for the resident or potential resident.

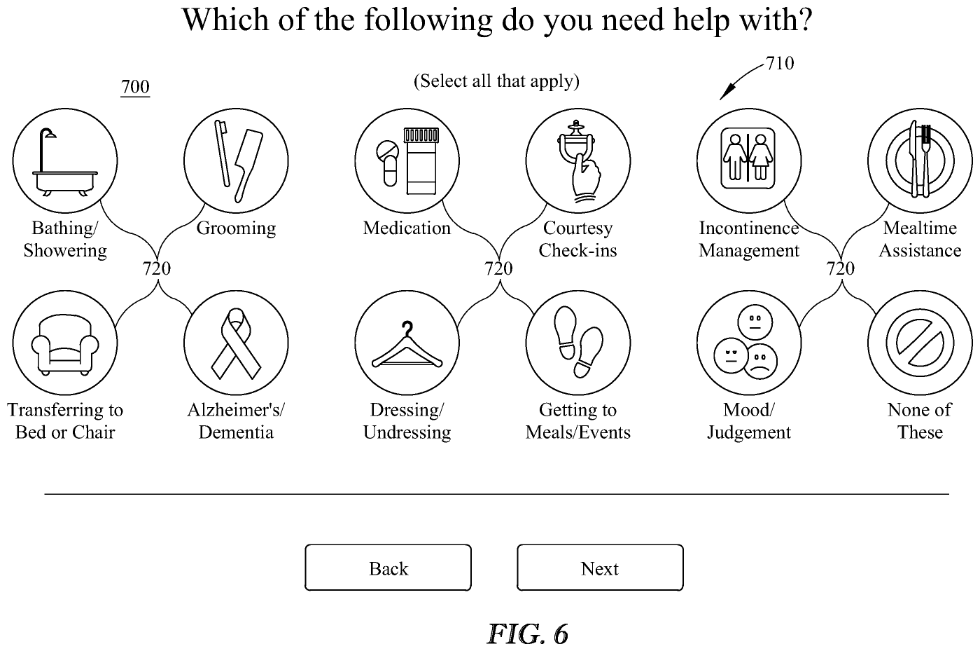

Referring to FIG. 6, and in some non-limiting examples, a user such as potential resident or family member of a potential resident may access a self-guided assessment model 700 using a self-assessment model interface 710 via a web browser or similar user interface to begin the assessment process. In some embodiments, the user may be prompted to specify which facility or community 1 there is an interest in, so that facility specific parameters may be used in the assessment model 700 when calculating pricing, as will be seen herein below. Self-assessment interface 710 permits a user to select from a plurality of acuity parameters 720, each having a plurality of configurable survey questions 730 associated therewith to assess the physical, emotional and mental health acuity of the resident and thereby determine the level of care required during residency.

As seen in FIG. 6, some exemplary acuity parameters 720 may include, but are not limited to, bathing and showering, grooming, medication requirements, courtesy check-in requirements, incontinence management, mealtime assistance, transferring to bed or chair, Alzheimer and dementia issues; dressing and undressing, getting to meals and events, and mood and judgment issues. The user may select as many of these acuity parameters they wish to include in the assessment model 700 to answer further survey questions 730 in the specific acuity categories. Once they are selected, the user is then guided through survey questions for each selected acuity parameter 720 to complete the assessment 700. Furthermore, since the user is required to specify a residential community 1 prior to using the assessment model 700, the survey questions 730 may be configured to comply with location specific regulatory variables. For example, where a state or other locale requires a facility to be aware of and report certain types of resident or staff incidents or actions, survey questions 730 may be designed to elicit responses required to comply with the regulations for that specific community 1 based upon the regulatory environment at that location.

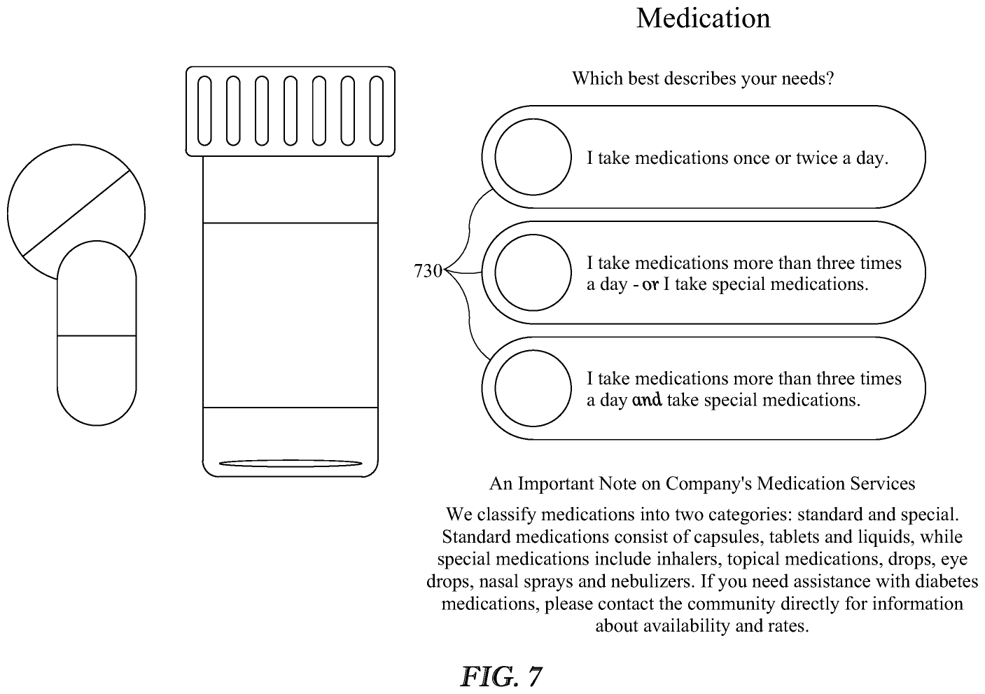

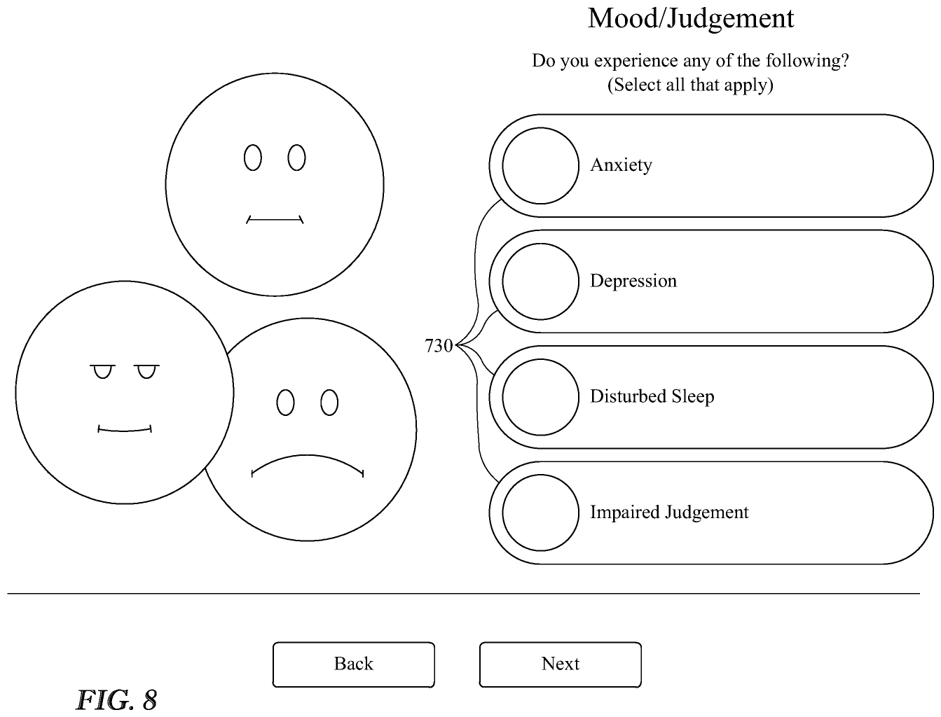

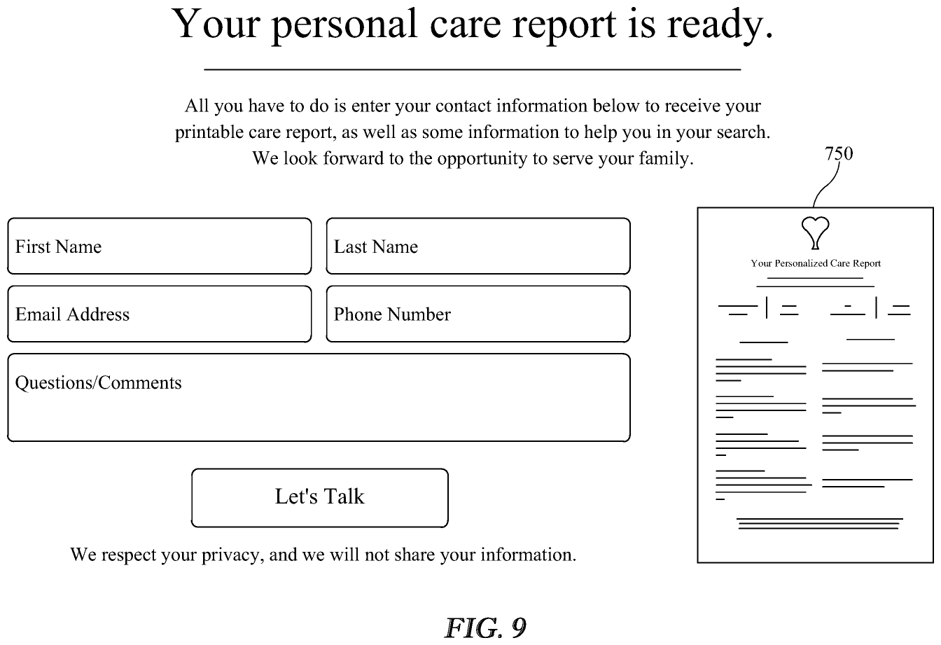

FIGS. 7 and 8 depict an exemplary series of survey questions 730 associated with medication requirements and mood and judgment issues 720, respectively. Further survey questions 730 are provided in a plurality of acuity parameters 720 relevant to resident care. Once the user selects the applicable categories of acuity provided in the survey questions, the assessment model 700 provides a care profile 750 to the user as shown in FIG. 9. In embodiments of the invention where a user is conducting a self-assessment model 700, the care profile 750 may be sent to the user via e-mail or similar electronic communication so that the system then has a user's contact information for follow-up. The care profile 750 in some aspects and embodiments includes an overall acuity level score for the person being assessed, so that an acuity baseline can be established for future comparison purposes, as well as details the tasks and care requirements of the potential resident. Additionally, in some embodiments the assessment 700 and concomitant care profile 750 may be sent to a sales associate or sales team assigned to the specific potential resident so that sales personnel are able to follow-up with the potential resident and know and understand their specific care requirements when discussing a service plan or room that meets the needs of that particular person.

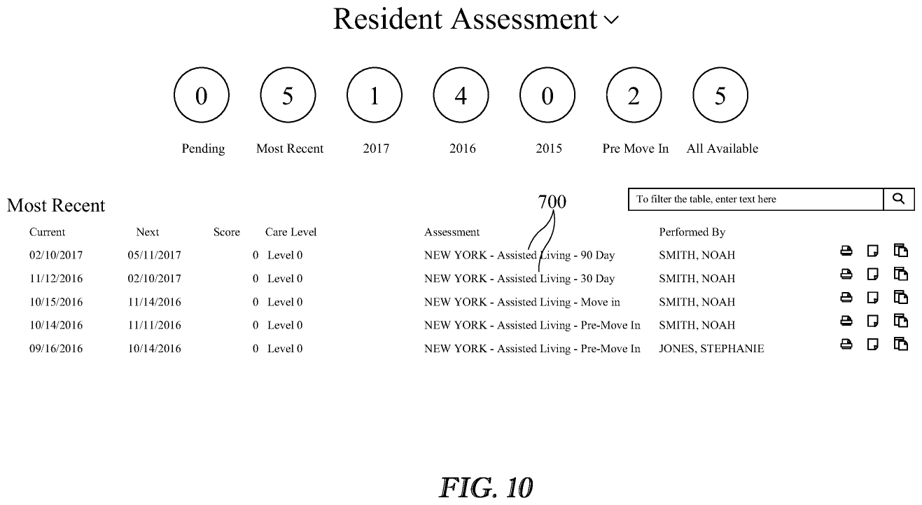

Referring now to FIGS. 10-12 and in accordance with further embodiments of the invention, a plurality of user interface pages 710 are shown for an assessment model 700 that may be utilized for a current resident of a residential community 1. Associated with each resident may be an assessment 700, or a plurality thereof, that is conducted by community 1 staff member at predetermined intervals, for example, every three months in the case of a typical resident. Alternatively, assessments 700 may be conducted at any time a resident has a change in acuity 720 that may occur from time to time due to an illness or other event. Each of these assessments for each individual resident are saved and stored for viewing at any time as depicted in FIG. 10. The assessment models 700 for residents within a community 1 may in some embodiments have a greater level of detail and therefore include additional and more detailed survey questions 730 than the above-disclosed self-assessment models 700.

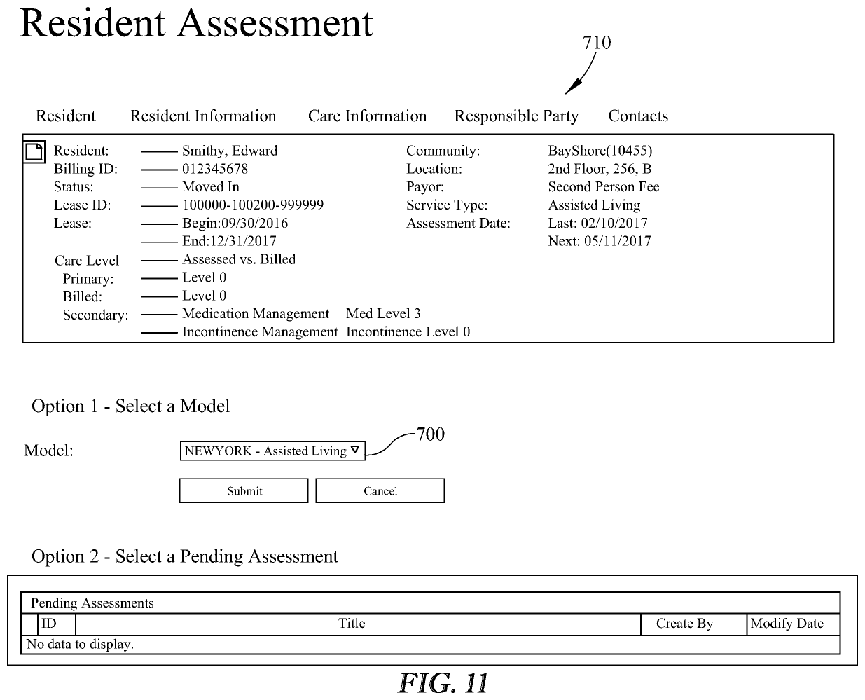

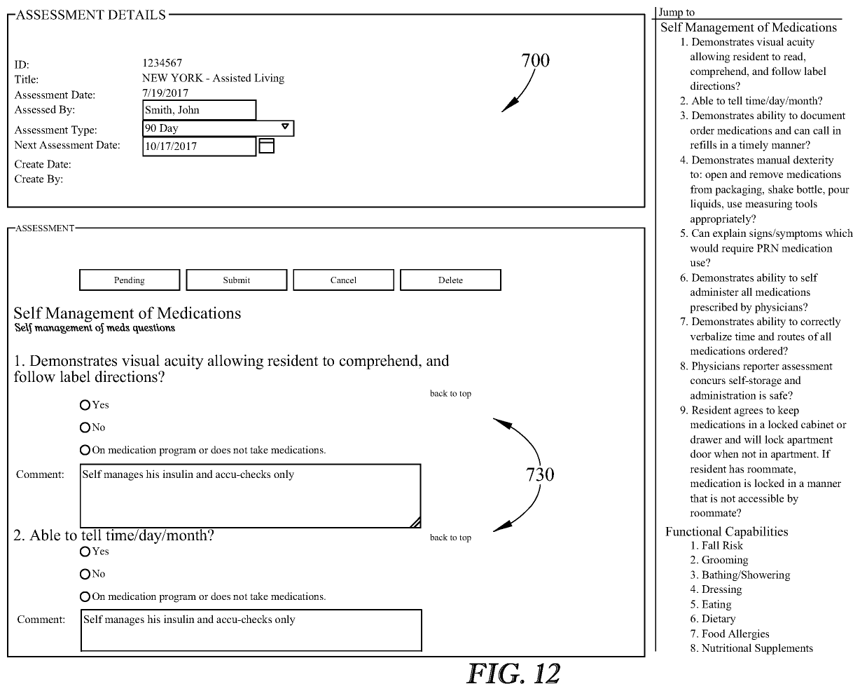

When community 1 staff or a user conducts an assessment, they choose the Resident Assessment tab that then opens an assessment page 710, depicted in FIG. 11. The user then provides or updates resident contact information and then chooses an assessment model, for example “assisted living” or “life guidance” (which is synonymous with “memory care”) to initiate the resident assessment. Once a model 700 is chosen, a plurality of survey questions 730 are then presented to the user as depicted partially in FIG. 12, who then steps through the assessment model 700 until completion. The resident acuity parameters 720 being assessed can range greatly and may differ based upon the model 700 selected. Exemplary assessment parameters 720 may include the ability to comprehend directions and manage the administration of their own medications, various functional capabilities such as fall risks, grooming, bathing, dressing, eating, dietary needs, allergies, nutritional supplements, hydration, hearing, vision, speech, medications, ordering medications, transfer ability, continence, assistive and adaptive device needs, various psycho-social capabilities such as socialization, spiritual, behavior, orientation, telephone use, housekeeping, laundry, pet care, and additional needs such as oxygen, diabetic care and assistance, skin care, wound care, catheterization, colostomy and ileostomy issues, weight loss and gain, periodic status checks, any monitoring required from outside services or specialists, and communication plans for residents with special communication needs.

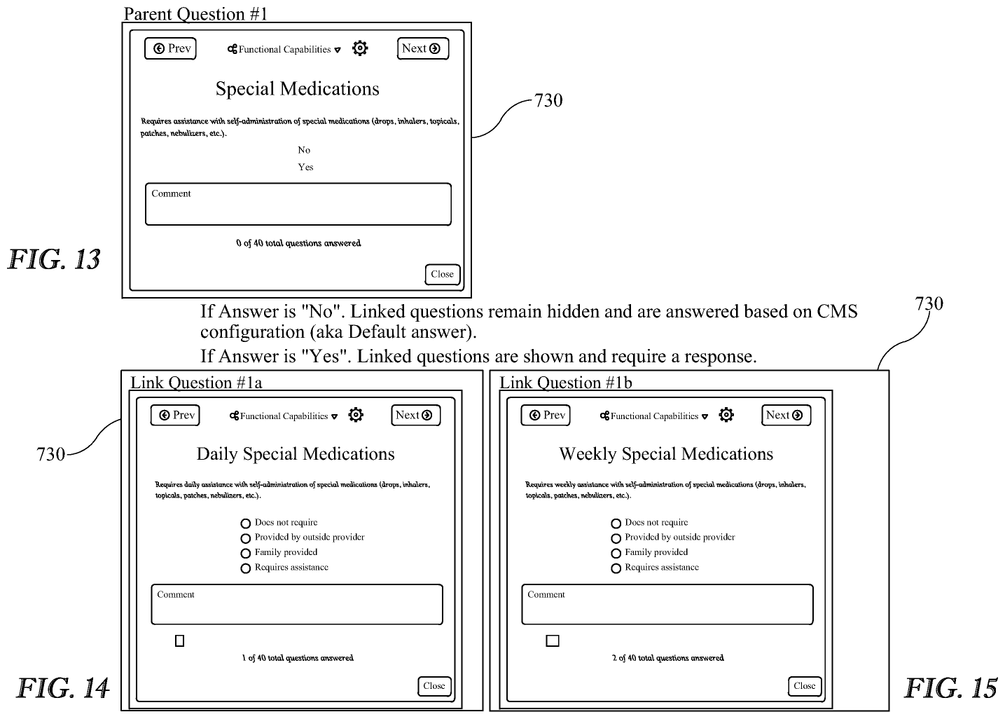

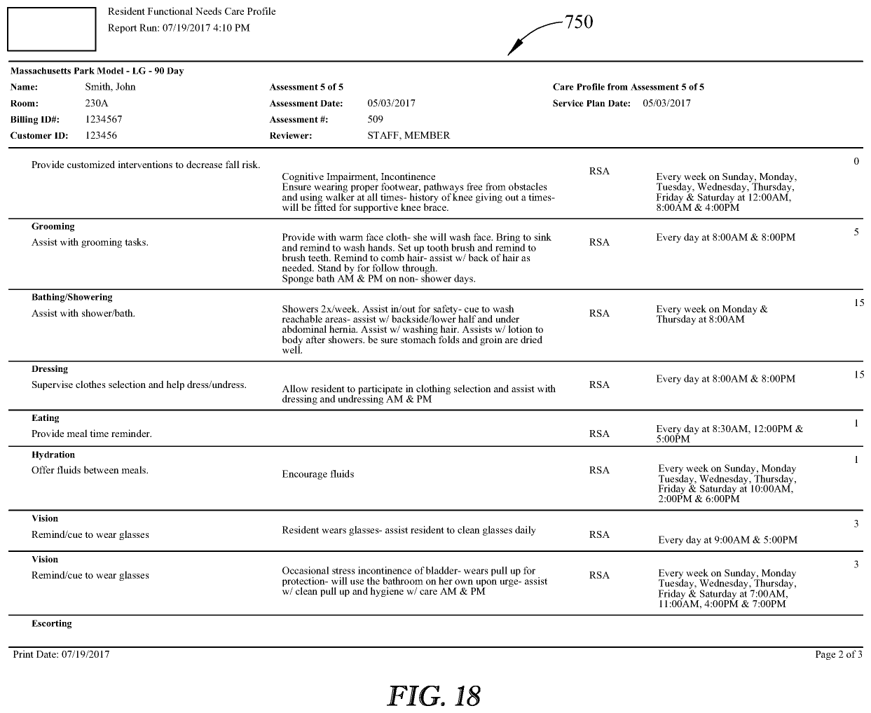

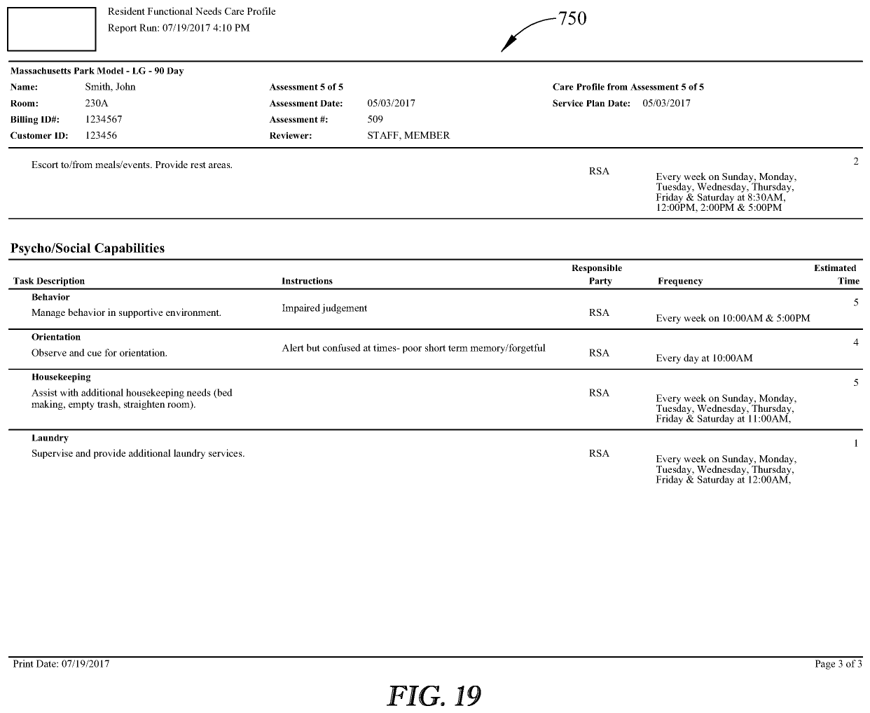

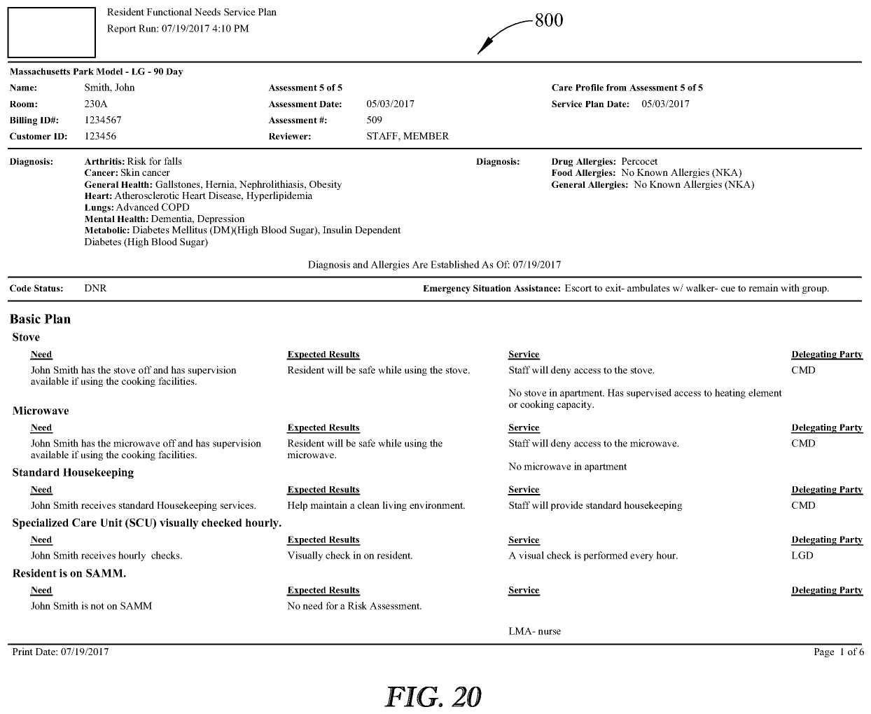

Referring to FIGS. 13-15, and in accordance with some aspects and embodiments of the invention, assessment survey questions 730 may be provided in the form of “nested” survey questions, wherein the answer to a first or “parent” survey question 730 dictate whether follow up linked questions 730 are necessary. In any exemplary embodiment as shown in FIG. 18, the assessment parameter “special medications” includes a parent survey question 730 that simply asks whether the resident is required to take any special medications. If the answer to that question is “No”, then the linked questions are skipped and the assessment 700 continues. If the answer is “Yes”, the linked questions shown in FIGS. 19 and 20 are then asked; whether there are daily or weekly special medications required, and what they are. This feature of the invention provides the user to avoid or skip linked or nested survey questions 730 that are not applicable to the resident being assessed, thereby providing for efficient assessment models 700 that avoid unnecessary survey questions 730.

Once a resident assessment 700 survey questions 730 are completed, an overall acuity score is generated that is indicative of the level of care and medication management required to support that resident in a given community 1. The overall acuity score in some aspects and embodiments is considered as a summation of effort and expense levels required to care for the resident being assessed. A higher acuity score is typically indicative of a resident requiring a higher level of assistance with daily activities, and/or requiring care that utilizes specialized care or personnel to administer the requisite assistance.

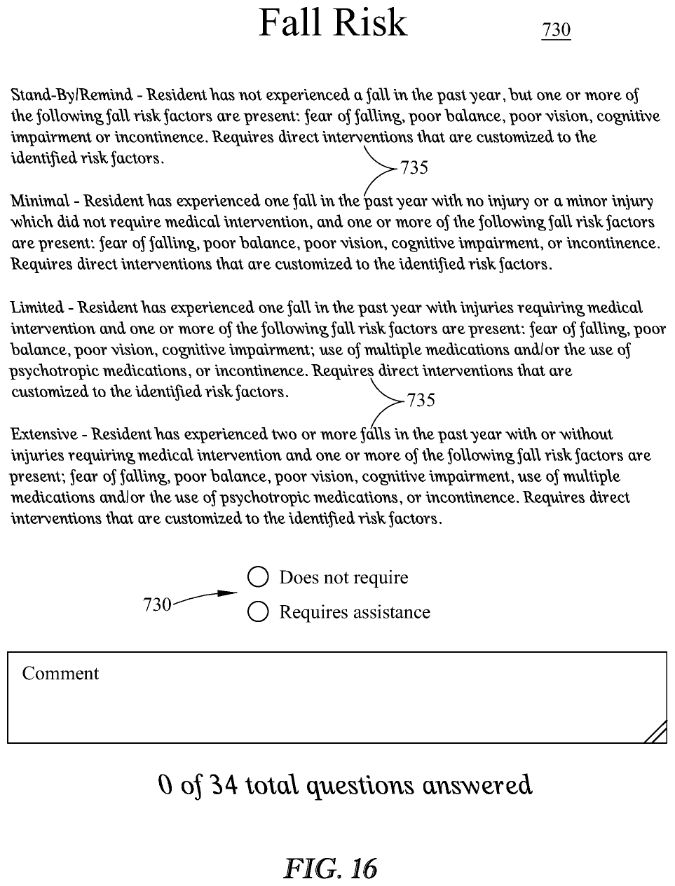

In some further aspects and embodiments of the invention as best seen in FIG. 16, each of the survey questions may be provided with a plurality of interpretive guidelines 735 that permit the assessor to accurately interpret the variable or factor being assessed in each question or area of the assessment 700. The interpretive guidelines 735 may incorporate aspects of regulatory requirements that are applicable to a given community 1 based on the legal or administrative jurisdiction in which it is operating, thereby enabling the residential community 1 staff to adequately prepare for any required actions, as well as provide a care profile 750 that incorporates all the tasks required of the community 1 staff to care for that particular resident. In the exemplary but non-limiting embodiment shown in FIG. 16, a plurality of interpretive guidelines 735 related to the assessment parameter 720 “Fall Risk” are provided to assist a user in correctly assessing the resident's level of assistance required to avoid a fall. In some aspects of the invention, the interpretive guidelines provided may incorporate statutory and/or regulatory requirements for residential facility operators, where applicable.

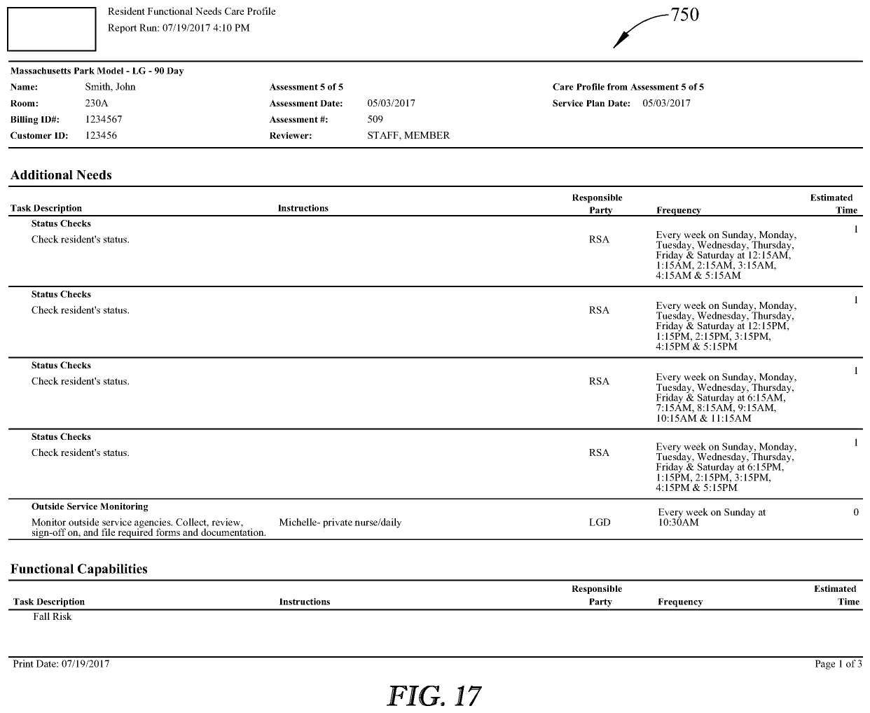

Referring now to FIGS. 17A-C, a care profile 750 is depicted, showing for a specific resident a list of care needs based on the assessment 700 as well as specific tasks scheduled at periodic intervals to meet those needs. In some aspects and embodiments care profiles 750 are specific to the state in which a community is located, since many states have different care requirements for residential community 1 residents based upon their needs. Care profiles 750 may be automatically generated based upon a completed assessment model 700, thereby providing a community 1 the ability to immediately schedule and prepare for the needs of its residents. Care profiles 750 are then provided to the care staff of a residential community so that the tasks included therein can be scheduled, assigned, and performed in an efficient manner. Since care profiles are generated on a resident-by-resident basis, and since care profiles may include similar tasks for similarly situated residents, in some aspects and embodiments the profiles 750 may be categorized and provided to the staff by resident, by staff responsible for certain residents, by task, by completed tasks, by incomplete tasks, or by scheduled task times. This feature of the invention may be automatically conducted by selecting a group of care profiles 750 and sorting them by the requisite category. Furthermore, these care profiles 750 and concomitant scheduled tasks may be provided to mobile or portable wireless devices carried by community 1 staff, so that completed tasks may be marked as such in real time, and incomplete tasks may be viewed by staff throughout the community 1.

In concert with care profiles 750 generated by the system 10 of the present invention, FIGS. 18A-F depict an exemplary but non-limiting service plan 800, which provides a cost baseline for the individual resident based upon the care profile 750 and the acuity level score. Service plans 800 may also be automatically generated based on care profiles 750 and assessments 700 and generally provide detailed tasks necessary to implement the care profile, and the costs associated with those tasks in that specific community 1 for the resident. In some embodiments, new care profiles 750 and service plans 800 are generated each time an assessment occurs, or each time a change in acuity level score occurs.

Referring now to FIGS. 19-21 the system and methods of the invention include an incident reporting system 900. FIG. 19 depicts a resident-specific user interface 910 for incident reporting, showing in large numbers for that resident the number of most recent incidents, the number of falls, the number of medication errors, property related incidents, accidents, injuries, illnesses and overall incidents. Below these indicators the incident interface 910 provides details for each recorded incident report 920 for that resident, including the date of the incident, the person reporting the incident, and a description of the incident. Selecting the “Incident” tab provides the user with the ability to report a resident incident utilizing a series of user interfaces that comprise an incident report 930 as depicted in FIGS. 20A-F. This incident report 930 includes a plurality of incident categories that can be used to detail any incident that may occur within a community 1. The incident report 930 provides an operator interface for preparing a report 930 that provides resident identifying information, and then details of any type of incident that may occur in a community 1 including, but not limited to, the time of the incident and the staff member recording it, fall details, medication details, altercation and exploitation details, property issues, elopement events, auto accidents, potential exposures, injuries, behavioral issues, illnesses, the outcome of any incident, emergency calls or contacts, and any follow-up tasks or contacts required for the individual incident.

Once a staff member prepares and saves an incident report 930 by supplying the information discussed herein above, an incident report 930 is automatically provided to management personnel and legal personnel in a residential community for further action appropriate to the specific incident. Furthermore, where an incident is of a predetermined type where a regulatory agency is required to be notified (depending upon the location of the residential community 1), the system 10 of the invention automatically provides to the user an applicable regulatory reporting form for completion and further action, such as legal review, according to the incident being reported. The system 10 may provide this incident report in a plurality of different ways, such as an automatically generated e-mail or other electronic or hard-copy format without departing from the scope of the invention.

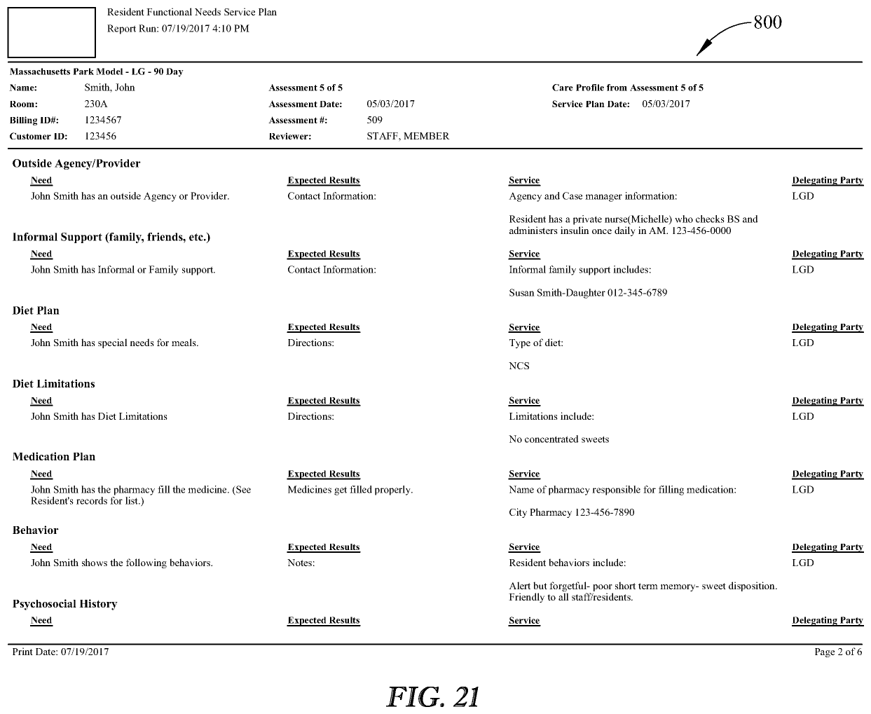

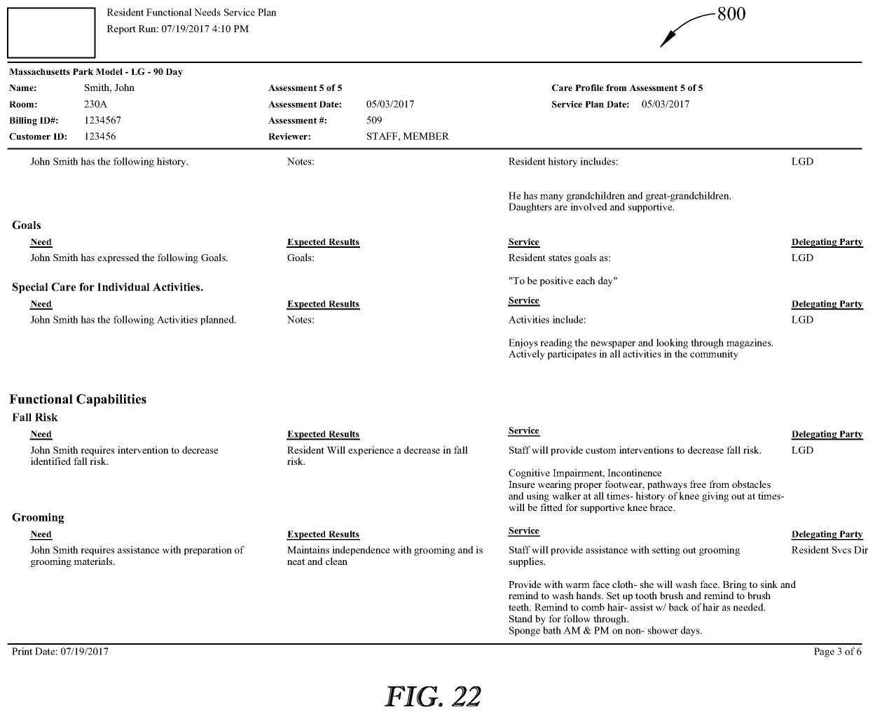

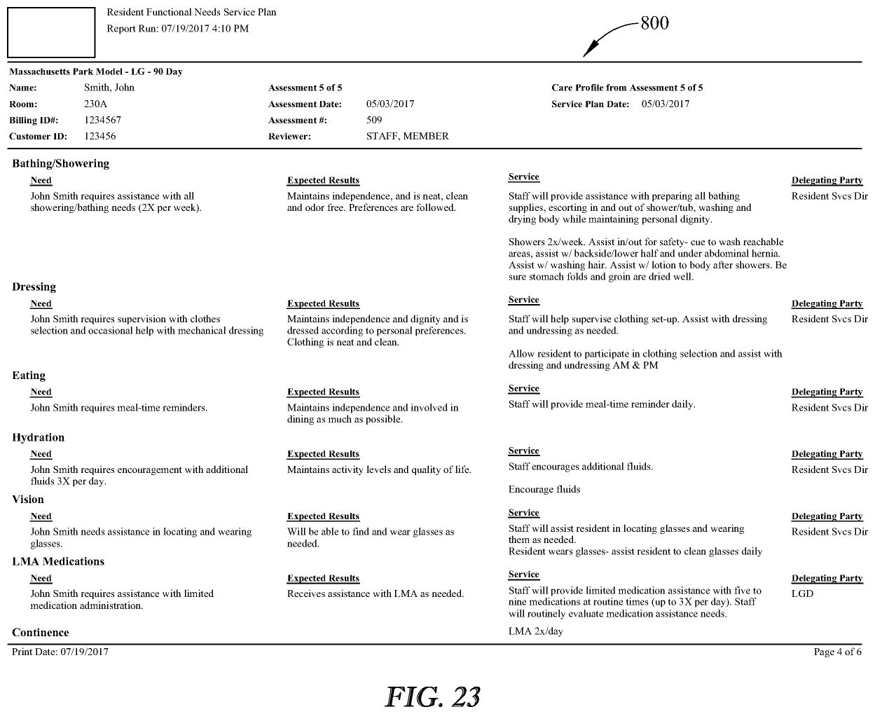

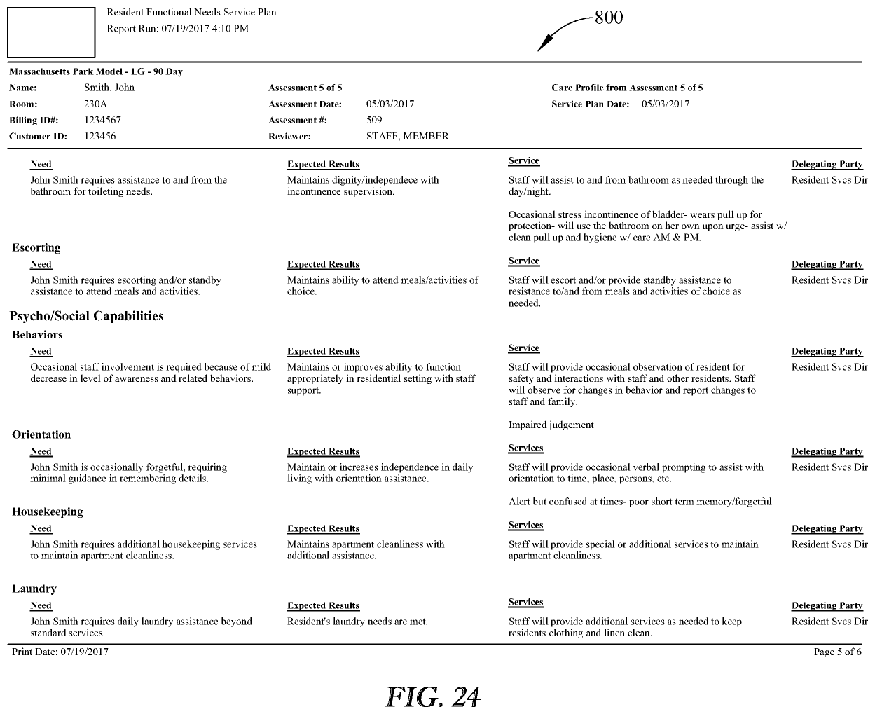

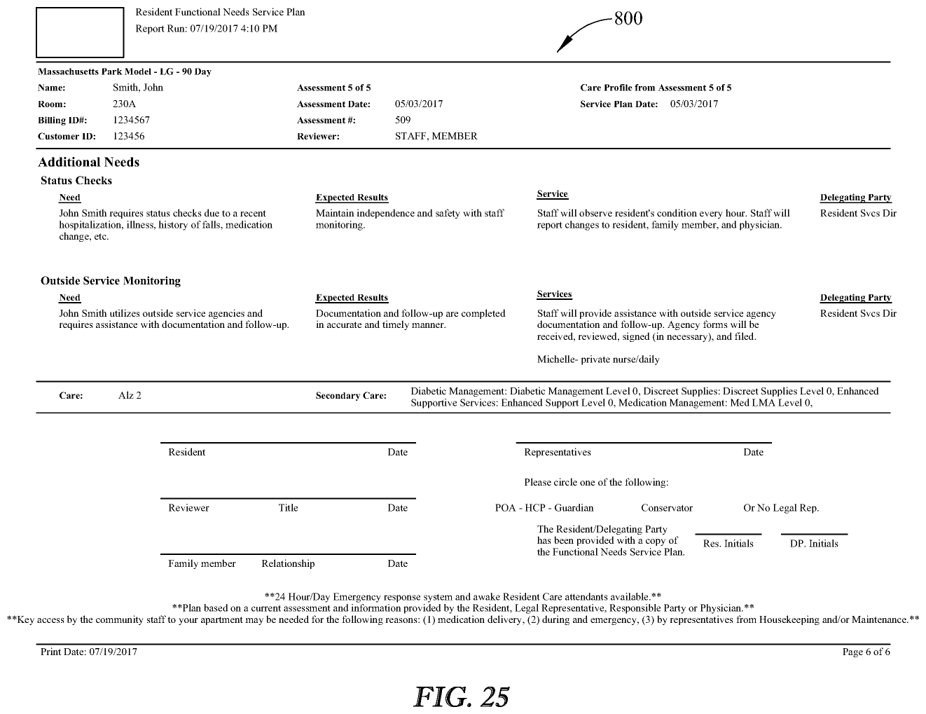

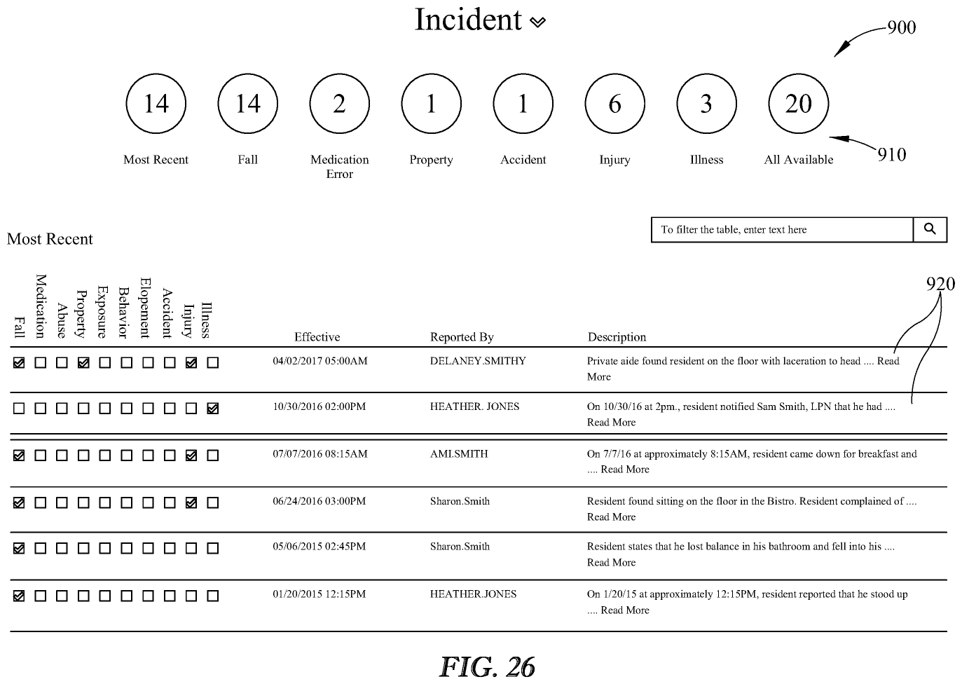

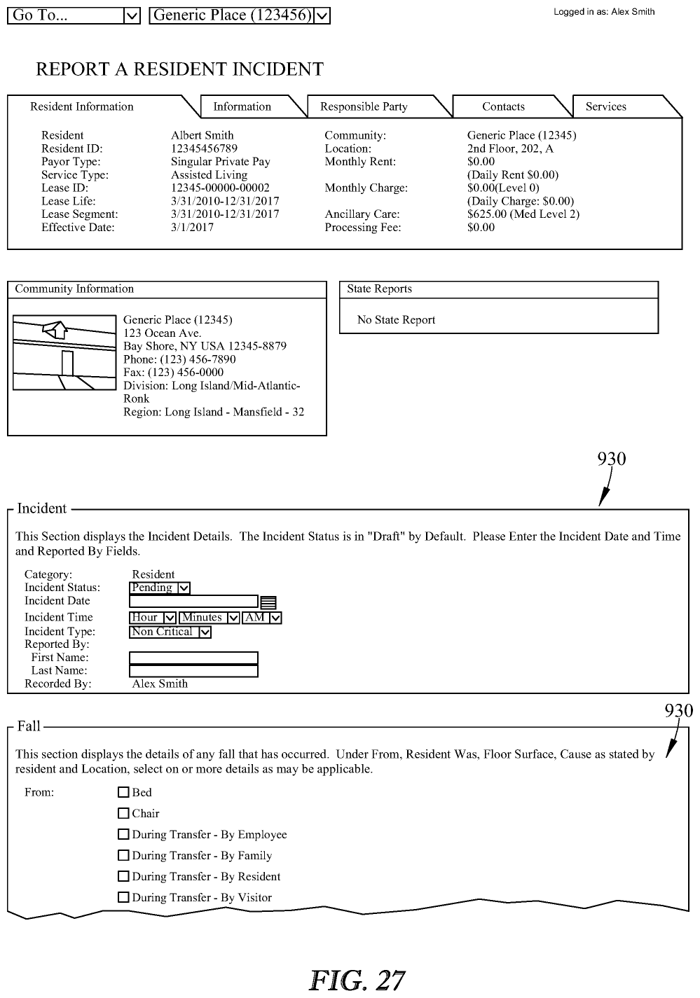

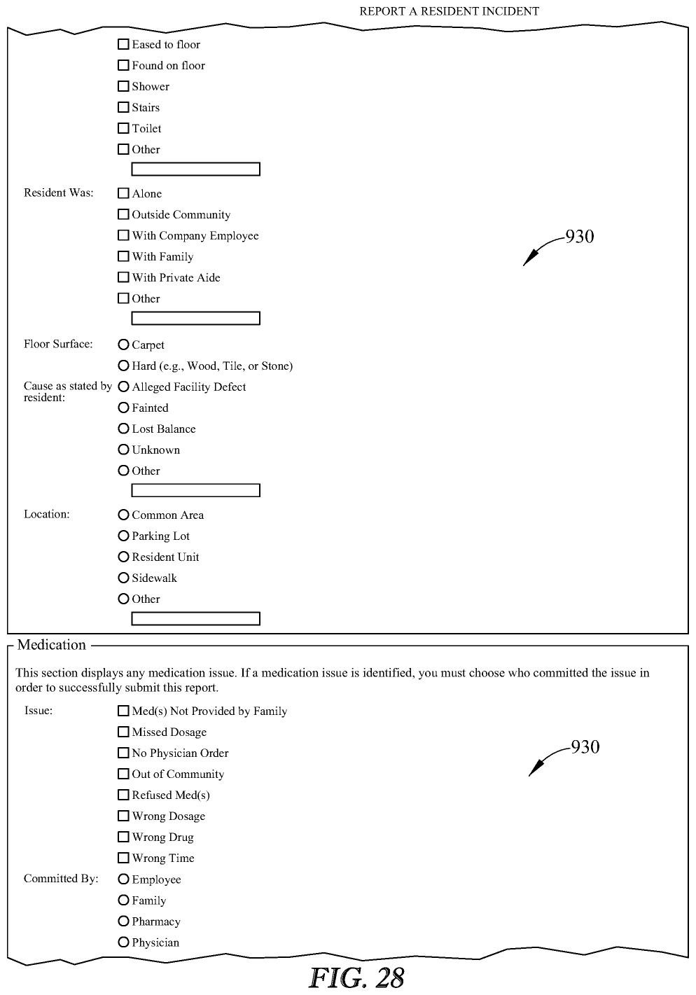

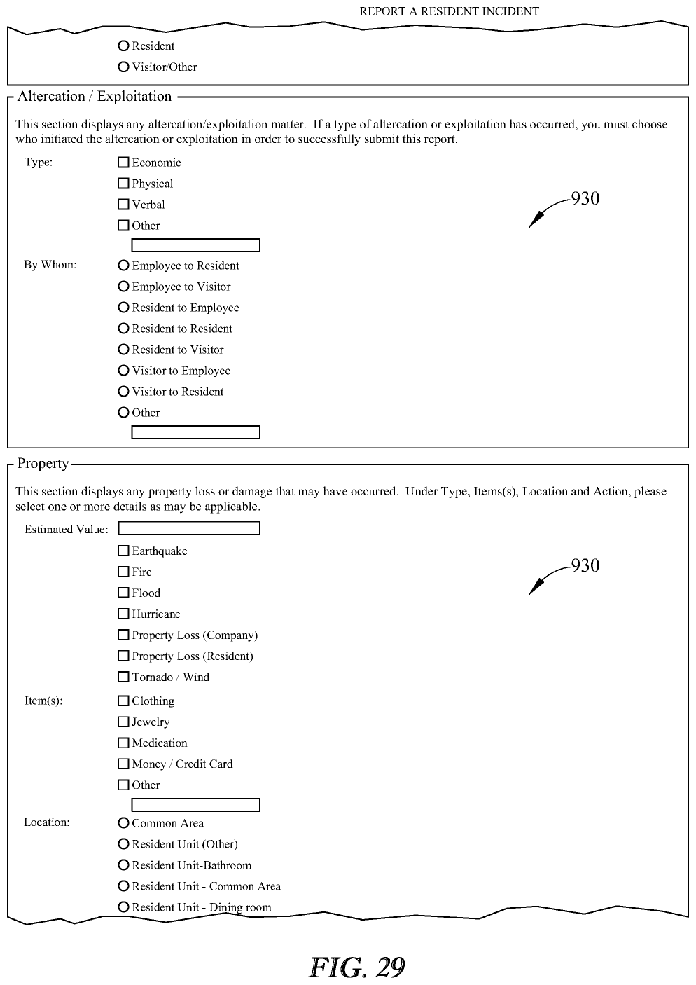

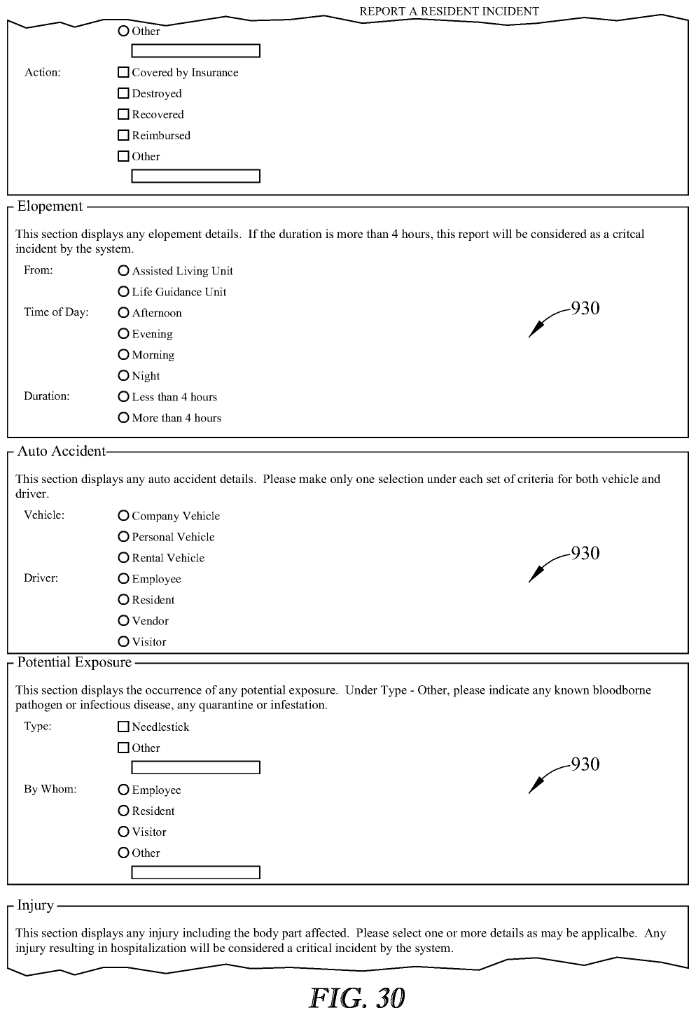

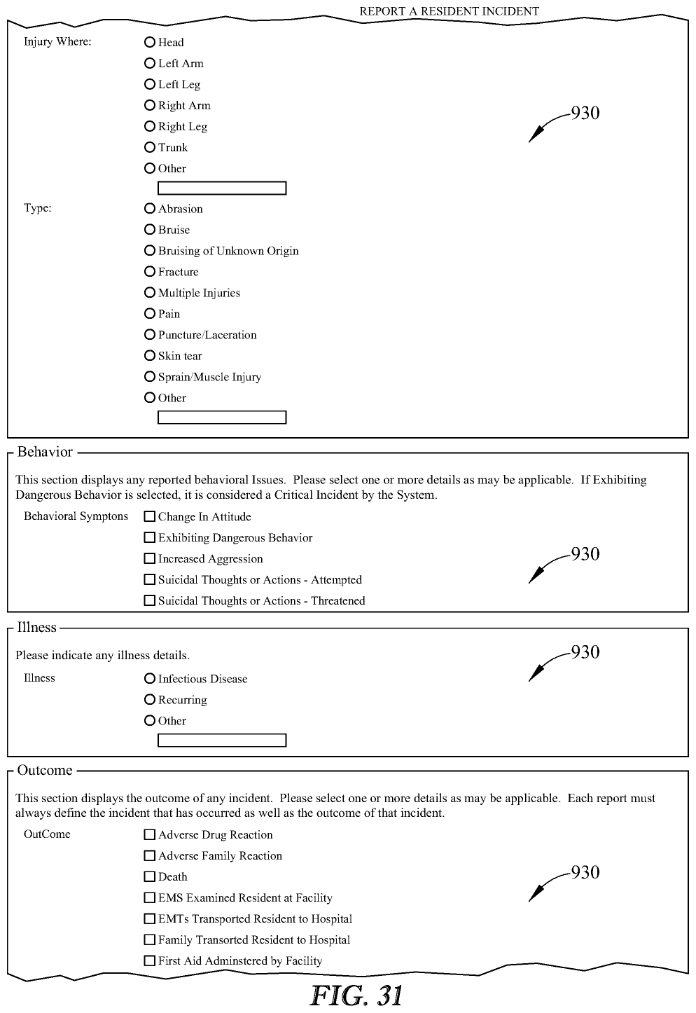

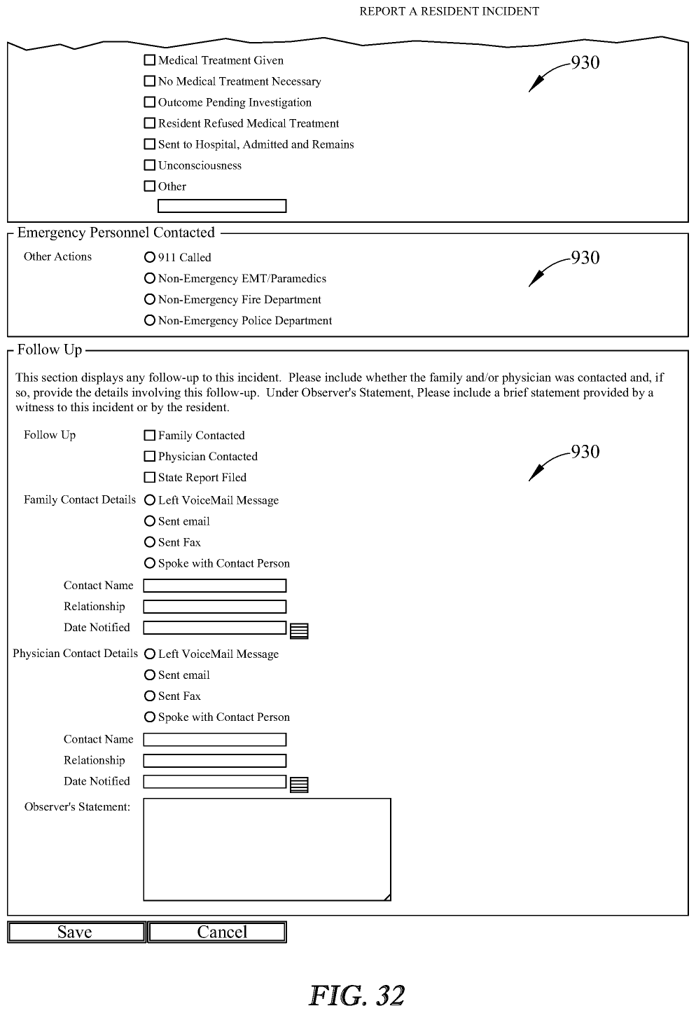

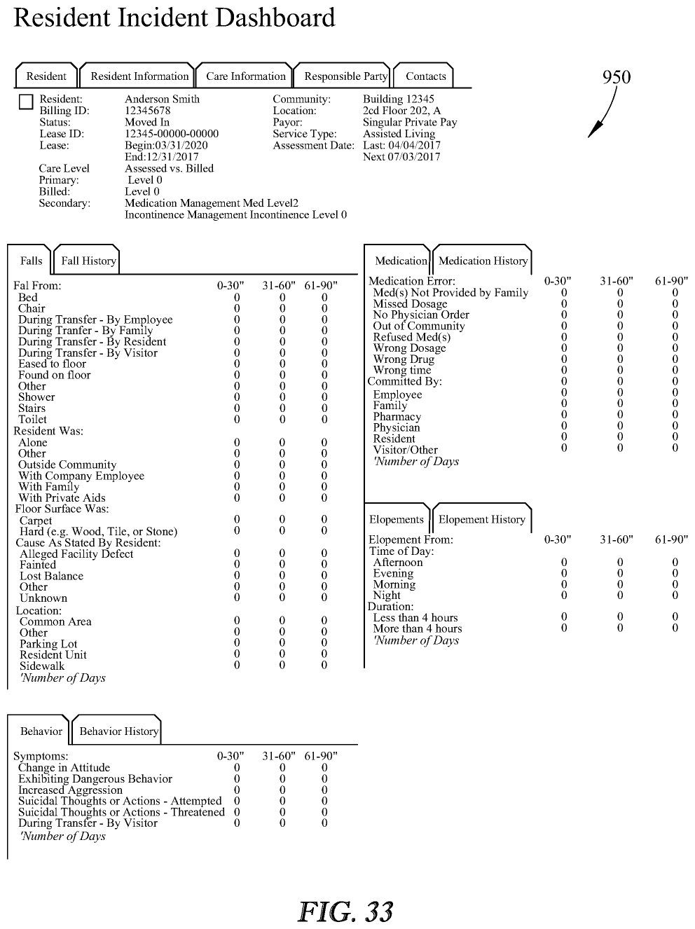

In other aspects and embodiments the system 10 of the present invention also provides a user with a Resident Incident Dashboard 950 as depicted in exemplary FIG. 21. Incident Dashboard 950 provides a detailed account and history of various categories of resident incidents over predetermined time periods, allowing a user to quickly assess whether incidents of any given type are occurring at an increased frequency. In this fashion, the invention provides the user, community 1 staff, or regional management the ability to determine potentially troubling trends for a resident before they become more severe, and take corrective action as necessary to avoid the occurrence of further incidents.

Other embodiments of the disclosure will be apparent to those skilled in the art from consideration of the specification and practice of the embodiments disclosed herein. It is intended that the specification and examples be considered as exemplary only, with a true scope and spirit of the invention and exemplary embodiments being indicated by the following claims.

What is claimed is:

1. A system for managing a plurality of resident communities having a plurality of rooms for occupancy, said system accessible by a plurality of individual users, the system comprising:
a memory device that stores a set of instructions and stores data relevant to said resident communities; and
at least one processor that executes the set of instructions to:
provide a user access login; and
provide an assessment system for each resident of a predetermined community, said assessment module comprising;
an health acuity tracking system assessment having health care tasks, task scheduling, resident contact information, service plans, and care profiles.

2. The system of claim 1 further comprising:
providing a resident management system for tracking resident data related to care preferences, end of life directives, personal and professional contacts, and emergency requirements.

3. The system of claim 1 further comprising:
providing an incident tracking system for tracking and documenting instances of resident accidents, health related incidents, theft incidents, and elopement incidents, said system having automatic managerial notifications based upon incident categories and severity.

4. The system of claim 3 further comprising:
selecting and providing to a user a required regulatory reporting form for predetermined incidents.

5. The system of claim 1 further comprising:
providing a system for tracking resident preferences in a plurality of categories including foods, beverages, entertainment, and spiritual preferences.

6. The system of claim 1 further comprising:
providing an assessment model;
formulating a service plan for each resident based on said assessment model; and
formulating a care profile for each resident.

7. The system of claim 6 further comprising:
providing an assessment model having a configurable survey comprising questions relevant to a residents needs, said survey being configured to conform with location specific regulatory variables; and
providing a set of interpretive guidelines for said survey questions to determine a resident acuity level score.

8. The system of claim 7 further comprising:
formulating a care profile for each resident including a list of care needs and tasks scheduled to meet those needs.

9. The system of claim 7 further comprising:
formulating a service plan based upon said care profile for each resident, said service plan providing pricing information based upon said residents' care profile and acuity level score.

10. The system of claim 9 further comprising:
updating said service plan at periodic intervals based upon changes to said care profile and said acuity level score.

11. The system of claim 10 further comprising:
providing said care profiles to a care staff, said care profiles being categorized by resident, by care staff, by completed tasks, by uncompleted tasks, and by suggested task times.

12. The system of claim 11 wherein said completed and uncompleted tasks are periodically reported to a resident services director.

13. The system of claim 10 further comprising:
a wireless communications link operably connected to said processor for providing said care profiles to a plurality of care staff wireless devices, whereby said care staff provides real time updates to care profile tasks.

14. The system of claim 1 further comprising:
providing a graphical interface accessible to individual users;
providing a self-guided assessment model;
formulating a service plan for each individual user based upon said self-guided assessment model;
formulating a pre-assessment for each user; and
providing said pre-assessment to each of said users and to a sales department.
